(19) United States
(12) Patent Application Publication
(10) Pub. No.: US 2022/0313099 A1
(43) Pub. Date: Oct. 6, 2022

(54) WEARABLE DEVICE, HEADSET DEVICE, AND A METHOD FOR OPERATING THE WEARABLE DEVICE

(71) Applicant: Advanced Semiconductor Engineering, Inc., Kaohsiung (TW)

(72) Inventors: Yu-Jung CHANG, Taoyuan (TW); Ming-Tau HUANG, Taoyuan (TW)

(73) Assignee: Advanced Semiconductor Engineering, Inc., Kaohsiung (TW)

(21) Appl. No.: 17/219,593

(22) Filed: Mar. 31, 2021

(51) Int. Cl. A61B 5/0245; G02C 11/00; A61B 5/024; A61B 5/28; A61B 5/318; A61B 5/00

(52) U.S. Cl. CPC A61B 5/0245; G02C 11/10; A61B 5/02405; A61B 5/02438; A61B 5/28; A61B 5/318; A61B 5/6803

(57) ABSTRACT

The present disclosure provides a wearable device. The wearable device includes a first sensing element configured to be disposed adjacent to a right ear of a user while the wearable device is worn by the user and a second sensing element configured to be disposed adjacent to a left ear of the user and coupled to the first sensing element while the wearable device is worn by the user. The second sensing element and the first sensing element are configured to sense a biological signal from the user. The wearable device also includes a reference electrode configured to reduce an interference to the biological signal. A headset device and a method for operating a wearable device is also provided in the present disclosure.

### BACKGROUND

### 1. Technical Field

The present disclosure generally relates to a wearable device. More particularly, the present disclosure relates to a wearable device capable of sensing biologically-relevant information.

### 2. Description of the Related Art

Monitoring biologically-relevant information helps determine a wide array of an individual's physiological characteristics. Integrating a monitoring device (such as a sensor) with a wearable device (such as a pair of glasses, an earpiece, and a watch) allows pertinent information to be collected in a continuous and nonintrusive manner, and thus has become increasingly popular.

### SUMMARY

In one or more embodiments, the present disclosure provides a wearable device. The wearable device includes a first sensing element configured to be disposed adjacent to a right ear of a user while the wearable device is worn by the user and a second sensing element configured to be disposed adjacent to a left ear of the user and coupled to the first sensing element while the wearable device is worn by the user. The second sensing element and the first sensing element are configured to sense a biological signal from the user. The wearable device also includes a reference electrode configured to reduce an interference to the biological signal.

In one or more embodiments, the present disclosure provides a headset device. The headset device includes a first sensing element configured to contact a first position of a user's head and receive a first electrical potential from the first position when the wearable device is worn by the user. The wearable device also includes a second sensing element configured to receive a second electrical potential from a second position of the user's body. The second position is different from the first position.

In one or more embodiments, the present disclosure provides a method for operating a wearable device. The method includes contacting a first conductive layer by a first position of a user's head to receive a first signal from the first position and contacting a second conductive layer by a second position of the user's hand to receive a second signal from the second position.

### BRIEF DESCRIPTION OF THE DRAWINGS

Aspects of the present disclosure are readily understood from the following detailed description when read with the accompanying figures. It should be noted that various features may not be drawn to scale. The dimensions of the various features may be arbitrarily increased or reduced for clarity of discussion.

FIG. 1A illustrates a three-dimensional (3D) view of a wearable device in accordance with some embodiments of the present disclosure.

FIG. 1B illustrates a cross-sectional view of a part of a wearable device in accordance with some embodiments of the present disclosure.

FIG. 1C illustrates a cross-sectional view of a part of a wearable device in accordance with some embodiments of the present disclosure.

FIG. 1D illustrates a cross-sectional view of a part of a wearable device in accordance with some embodiments of the present disclosure.

FIG. 1E illustrates a cross-sectional view of a part of a wearable device in accordance with some embodiments of the present disclosure.

FIG. 1F illustrates a cross-sectional view of a part of a wearable device in accordance with some embodiments of the present disclosure.

FIG. 2 illustrates the wearable device of FIG. 1A being used in accordance with some embodiments of the present disclosure.

FIG. 3A illustrates a 3D view of an ear hook on a part of a wearable device in accordance with some embodiments of the present disclosure.

FIG. 3B illustrates a cross-sectional view of a part of a wearable device in accordance with some embodiments of the present disclosure.

FIG. 4 illustrates a 3D view of a wearable device in accordance with some embodiments of the present disclosure.

FIG. 5 illustrates a 3D view of a wearable device in accordance with some embodiments of the present disclosure.

Common reference numerals are used throughout the drawings and the detailed description to indicate the same or similar elements. The present disclosure will be more apparent from the following detailed description taken in conjunction with the accompanying drawings.

### DETAILED DESCRIPTION

The following disclosure provides for many different embodiments, or examples, for implementing different features of the provided subject matter. Specific examples of components and arrangements are described below. These are, of course, merely examples and are not intended to be limiting. In the present disclosure, reference to the formation of a first feature over or on a second feature in the description that follows may include embodiments in which the first and second features are formed in direct contact, and may also include embodiments in which additional features may be formed between the first and second features, such that the first and second features may not be in direct contact. Besides, the present disclosure may repeat reference numerals and/or letters in the various examples. This repetition is for simplicity and clarity and does not in itself dictate a relationship between the various embodiments and/or configurations discussed.

Embodiments of the present disclosure are discussed in detail below. It should be appreciated, however, that the present disclosure provides many applicable concepts that can be embodied in a wide variety of specific contexts. The specific embodiments discussed are merely illustrative and do not limit the scope of the disclosure.

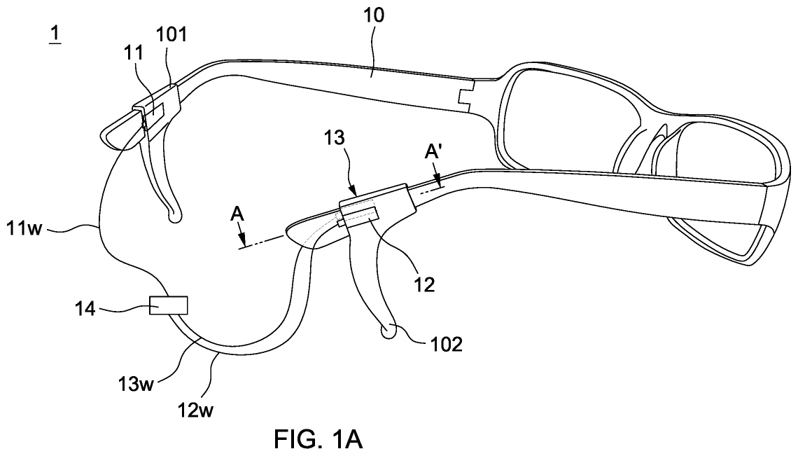

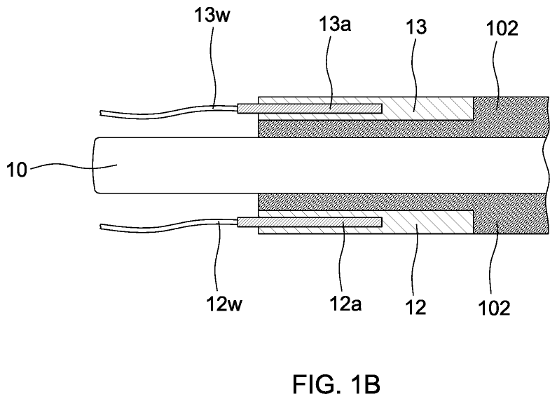

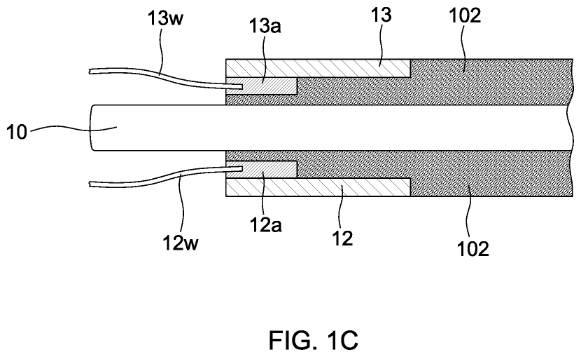

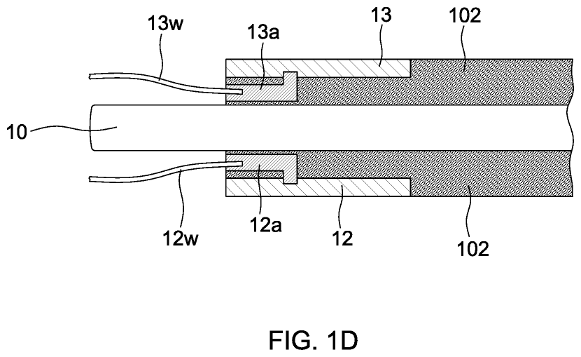

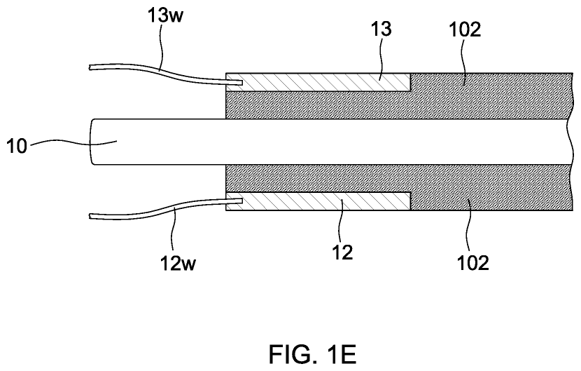

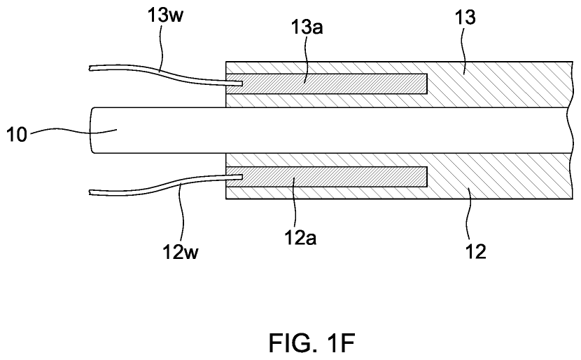

FIG. 1A illustrates a 3D view of a wearable device 1 in accordance with some embodiments of the present disclosure. Examples of the wearable device 1 include a pair of glasses, a pair of smart glasses, and so on. In some examples, the wearable device 1 may be other head-mounted devices, such as a headset (e.g., a virtual reality (VR) headset and an augmented reality (AR) headset), a headphone, an earphone, an earpiece, or a face mask. While the wearable device 1 is shown as a pair of glasses in FIG. 1A, the wearable device 1 is used to illustrate a piece of equipment that a user wear in or near their ear(s).

The wearable device 1 has one or more sensing elements. In some examples, the sensing element may be located on a supporting element or a support of the wearable device 1. In some examples, the sensing element may be attached to a user's head by the support. In some examples, the sensing element may be located on the temples (or hinges) 10 of the wearable device 1. In some examples, the sensing element may be attached to a user's head by the temples 10 of the wearable device 1. In some examples, the sensing element may be adjacent to or contact with a user's head when the wearable device 1 is worn by the user. In some embodiments, the wearable device 1 has a pair of ear hooks (including a portion 101 and a portion 102) and the one or more sensing elements are provided on the ear hooks. In some embodiments, the sensing element may be attached to a user's head by the portion 101 and the portion 102. In some examples, the sensing element may include conductive layers 11, 12, and 13.

As shown in FIG. 1A, the ear hooks are mounted on the temples (or hinges) 10 of the wearable device 1. When the wearable device 1 is worn by the user, the portion 101 and the portion 102 are on opposite sides with respect to the user's heart. The conductive layer 11 is provided on the portion 101, and the conductive layers 12 and 13 are provided on the portion 102. For example, the conductive layer 11 may be on the user's left side and the conductive layers 12 and 13 may be on the user's right side. In some embodiments, the conductive layers 12 and 13 are provided on opposite sides of the portion 102. In some embodiments, the conductive layers 12 and 13 are physically disconnected by the portion 102.

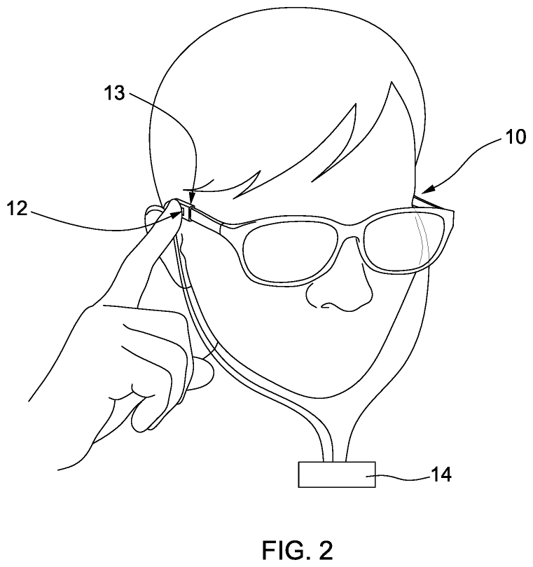

In some embodiments, each of the conductive layers 11, 12, and 13 may be used to contact several body parts of the user and receive (or transmit) signals (e.g., electrical signals) or voltages (e.g., electrical potentials) from the user. In some embodiments, each of the conductive layers 11, 12, and 13 may be coupled to one another or to a sensor module 14. The operations of the wearable device 1 may be further described with respect to FIG. 2.

In some embodiments, the signals received from (or transmitted to) the conductive layers 11, 12, and 13 may be transmitted to (or received from) the sensor module 14 though, for example, conductive wires 11w, 12w, and 13w, respectively. In some embodiments, the conductive wires 11w, 12w, and 13w may be omitted, and the conductive layers 11, 12, and 13 may be coupled to the sensor module 14 by wireless communication technology, such as Bluetooth.

In some embodiments, the signals may be further processed by the sensor module 14 to determine a biological parameter of the user, such as a pulse travel time (PTT), an electroencephalogram (EEG), electrocardiogram (ECG), electromyogram (EMG), electrooculogram (EOG), galvanic skin response (GSR), sweat composition, pH, heart rate variability (HRV), or other biologically-relevant information associated with the user.

In some embodiments, each of the conductive layers 11, 12, and 13 may include a conductive material, such as a metal or metal alloy. Examples include gold (Au), silver (Ag), aluminum (Al), copper (Cu), or an alloy thereof. In some embodiments, each of the conductive layers 11, 12, and 13 may include a flexible conductive material, for example, a conductive silicone, a thermal conductive silicone, a conductive rubber, a conductive sponge, a conductive fabric, or a conductive fiber. Each of the conductive layers 11, 12, and 13 may be soft and flexible enough for the user to wear for an extended time period without feeling uncomfortable.

In some embodiments, each of the conductive layers 11, 12, and 13 may be or may include an electrode, a thermistor, or a capacitive sensor. In some embodiments, each of the conductive layers 11, 12, and 13 may be or may include a conductive trace, a conductive pad or a conductive foil. In some embodiments, the shape and the dimension of each of the conductive layers 11, 12, and 13 are not limited to the particular embodiment as illustrated in FIG. 1A. For example, each of the conductive layers 11, 12, and 13 may cover the entire surfaces of the ear hooks. For example, the conductive layer 11 may extend between two ends of the inner surface of the portion 101. For example, the conductive layer 13 may extend between two ends of the inner surface of the portion 102. For example, the conductive layer 12 may extend between two ends of the outer surface of the portion 102.

FIG. 1B illustrates a cross-sectional view of a part of a wearable device in accordance with some embodiments of the present disclosure. In some embodiments, the configuration and arrangement of the conductive layer in FIG. 1B may be applied in the wearable device 1 in FIG. 1A. The conductive layer 12 has a surface exposed from an exterior surface (i.e., a side opposite to a skin-facing side) of the portion 102 of the ear hooks, and the conductive layer 13 has a surface exposed from an interior surface (i.e., a skin-facing side) of the portion 102 of the ear hooks. Similarly, although not illustrated in FIG. 1B, the conductive layer 11 as shown in FIG. 1A may have a surface exposed from an interior surface (i.e., a skin-facing side) of the portion 101 of the ear hooks.

In some embodiments, the exposed surface of the conductive layer 12 may be substantially coplanar with the exterior surface of the portion 102 of the ear hooks. In some embodiments, the exposed surface of the conductive layer 13 may be substantially coplanar with the interior surface of the portion 102 of the ear hooks. In some embodiments, the exposed surface of the conductive layer 11 may be substantially coplanar with the interior surface of the portion 101 of the ear hooks. In some embodiments, each of the conductive layers 11, 12, and 13 may protrude from the portion 101 and the portion 102 of the ear hooks, which may help to increase the areas of contact with the skin, and may enhance the signal transmission.

In some embodiments, each of the portion 101 and the portion 102 of the ear hooks may include a dielectric material. In some embodiments, each of the portion 101 and the portion 102 of the ear hooks may include a non-conductive material or an insulating material. In some embodiments, each of the portion 101 and the portion 102 of the ear hooks may include, for example, rubber, silicon, sponge, or other suitable material such as an elastic material, a soft material, or a flexible material. The portion 101 and the portion 102 of the ear hooks may each be soft and flexible enough for the user to wear for an extended time period without feeling uncomfortable.

In some embodiments, each of the conductive layers 11, 12, 13 may be physically separated from the temples 10 of the wearable device 1 by the material of the ear hooks. In some embodiments, each of the conductive layers 11, 12, 13 may be electrically isolated from the temples 10 of the wearable device 1 by the material of the ear hooks.

The conductive layer 12 may have a conductive element 12a embedded in the conductive layer 12. In some embodiments, the conductive element 12a may be covered or surrounded by the conductive layer 12. In some embodiments, the conductive element 12a may protrude or extend into the conductive layer 12. In some embodiments, a shape of the conductive element 12a may be conformal to a shape of the conductive layer 12. In some embodiments, the conductive element 12a may be in contact with the conductive layer 12.

In some embodiments, the conductive element 12a may be partially covered or surrounded by the conductive layer 12. For example, the conductive element 12a may be partially exposed from the conductive layer 12. For example, an end of the conductive element 12a may be exposed from the conductive layer 12 to connect with the conductive wire 12w.

In some embodiments, the conductive element 12a may be or may include an electrode, a thermistor, or a capacitive sensor. In some embodiments, the conductive element 12a may be or may include a conductive trace, a conductive pad, or a conductive foil. In some embodiments, the shape and the dimension of the conductive element 12a are not limited to the particular embodiment as illustrated in FIG. 1B.

In some embodiments, the conductive element 12a may have a material as listed above for the conductive layer 12. In some embodiments, the conductive element 12a and the conductive layer 12 may have the same material. In some embodiments, the conductive element 12a and the conductive layer 12 may have different materials. In some embodiments, a conductivity of the conductive layer 12 may be different from that of the conductive element 12a. In some embodiments, the conductivity of the conductive element 12a may be greater than that of the conductive layer 12. In some embodiments, a resistivity of the conductive layer 12 may be different from that of the conductive element 12a.

In some embodiments, the conductive element 12a may be electrically coupled with the conductive layer 12. While being worn by the user, the conductive layer 12 is closer than the conductive element 12a to the user's skin. The signals collected by the conductive layer 12 may be transmitted to the embedded conductive element 12a, which provides a low-resistance/high-conductance transmission path for the signals.

Furthermore, a part of the conductive element 12a extends into the conductive layer 12. This arrangement enlarges the contact area between the conductive element 12a and the conductive layer 12. As such, the total resistance can be relatively low and thus the quality of the signals can be improved.

Similarly, the conductive layer 13 may have a conductive element 13a embedded in the conductive layer 13, and the conductive layer 11 may have a conductive element 11a embedded in the conductive layer 11. The conductive element 13a and the conductive element 11a may be similar with the conductive element 12a and the details thereof will not be repeated hereafter.

FIG. 1C illustrates a cross-sectional view of a part of a wearable device in accordance with some embodiments of the present disclosure. The part of the wearable device in FIG. 1C is similar to the part of the wearable device in FIG. 1B except for the differences described below.

In some embodiments, the conductive element 12a may be not embedded in the conductive layer 12. A surface of the conductive element 12a may be in contact with a surface of the conductive layer 12. A surface of the conductive element 12a may be disposed adjacent to a surface of the conductive layer 12. A surface of the conductive element 12a may connect with a surface of the conductive layer 12. The conductive element 12a may be side-by-side with the conductive layer 12.

In some embodiments, an end of the conductive element 12a may be exposed from the conductive layer 12 to connect with the conductive wire 12w. In some embodiments, the exposed surface of the conductive element 12a may be substantially coplanar with the conductive layer 12.

Similarly, the conductive element 13a and the conductive element 11a may be similar with the conductive element 12a and the details thereof will not be repeated hereafter.

FIG. 1D illustrates a cross-sectional view of a part of a wearable device in accordance with some embodiments of the present disclosure. The part of the wearable device in FIG. 1D is similar to the part of the wearable device in FIG. 1B except for the differences described below.

In some embodiments, the conductive element 12a has an extending portion protruding into the conductive layer 12 and a main portion connected with the conductive wire 12w. The main portion of the conductive element 12a may be spaced apart from the conductive layer 12. A part of the portion 102 may be disposed between the main portion of the conductive element 12a and the conductive layer 12. The extending direction of the extending portion may be different from the extending direction of the main portion of the conductive element 12a. In some embodiments, the extending portion and the main portion of the conductive element 12a may have the same material. In some embodiments, the extending portion and the main portion of the conductive element 12a may have different materials.

Similarly, the conductive element 13a and the conductive element 11a may be similar with the conductive element 12a and the details thereof will not be repeated hereafter.

FIG. 1E illustrates a cross-sectional view of a part of a wearable device in accordance with some embodiments of the present disclosure. The part of the wearable device in FIG. 1E is similar to the portion of the wearable device in FIG. 1B except for the differences described below.

In some embodiments, the conductive elements embedded in the conductive layers 11, 12, and 13 may be omitted. An end of the conductive layer 12 may be connected with the conductive wire 12w to couple to a sensor module or other external device. By excluding the embedded conductive elements, the cost of the wearable device 1 can be relatively low. By adjusting the conductivity of the conductive layer 12, the quality of the electrical signals transmitted in the wearable device may still be maintained at an acceptable level. Similarly, the conductive layer 13 and the conductive layer 11 may be similar with the conductive layer 12 and the details thereof will not be repeated hereafter.

FIG. 1F illustrates a cross-sectional view of a part of a wearable device in accordance with some embodiments of the present disclosure. The part of the wearable device in FIG. 1F is similar to the portion of the wearable device in FIG. 1B except for the differences described below.

In some embodiments, the ear hook and the conductive layers 11, 12, and 13 may have the same material. In other words, the entire ear hook may be an electrode. Therefore, the conductive layers 11, 12, and 13 may be in direct contact with the temples 10 of the wearable device 1. FIG. 2 illustrates the wearable device 1 of FIG. 1A being used in accordance with some embodiments of the present disclosure.

In some embodiments, the conductive layer 11 may contact the user to receive the biological signals, the conductive layer 12 may contact the user to receive the biological signals, and the conductive layer 13 may contact the user to provide a reference for the biological signals (such as ECG signals), or may otherwise be used to reject or remove noise from the biological signals. For example, the conductive layer 13 may contact the user to reduce an interference to the biological signals of the user. For example, the sensor module may include a biological signal amplifier electrically connected with the referenced electrode to reduce a common-mode interference. This may result in more accurate readings (or processing) of the biological signals.

As another example, the reference conductive layer (such as the conductive layer 13) may be omitted.

In some embodiments, the conductive layer 11 and the conductive layer 12 may be used to form a part of a signal loop passing through the user's heart. For example, the conductive layer 11 and the conductive layer 12 may be used to form a part of an ECG lead.

For example, when the user wears the wearable device 1 of FIG. 1A, the conductive layer 11 may contact the user's left ear (or a near portion thereof), and the user may contact the conductive layer 12 by the right hand. Thus, the conductive layer 11 and the conductive layer 12 may form a signal loop between the left ear and the right hand.

The user may contact the conductive layer 11 by a first part of their body and contact the conductive layer 12 by a second part of their body depending on where the conductive layer 11 and the conductive layer 12 are located on the wearable device 1. The first part may include any location or position of the user's body on one side and the second part may include any location or position of the user's body on the opposite side. The first part may be different from the second part. For instance, the user may contact the conductive layer 11 by a first part of their body on the left side and contact the conductive layer 12 by a second part of their body on the right side. For instance, the user may contact the conductive layer 11 by a first part of their body on the right side and contact the conductive layer 12 by a second part of their body on the left side.

For example, in some embodiments where the conductive layer 11 contacts the user's right ear (or a near portion thereof), the user may contact the conductive layer 12 by the left hand. Thus, the conductive layer 11 and the conductive layer 12 may form a signal loop between the right ear and the left hand.

In some embodiments, the conductive layer 11 and the conductive layer 12 may be provided on the same side. For example, in some embodiments where the conductive layer 11 contacts the user's left ear (or a near portion thereof) and the conductive layer 12 is also provided on the left side of the ear hook, the user may contact the conductive layer 12 by the right hand. Thus, the conductive layer 11 and the conductive layer 12 may form a signal loop between the left ear and the right hand.

Similarly, in some embodiments where the conductive layer 11 contacts the user's right ear (or a near portion thereof) and the conductive layer 12 is also provided on the right side of the ear hook, the user may contact the conductive layer 12 by the left hand. Thus, the conductive layer 11 and the conductive layer 12 may form a signal loop between the right ear and the left hand.

The positions of each of the conductive layers 11, 12, and 13 may be designed to make it easier for a user to place a part of the hand (e.g., finger) in contact with different conductive layers. Therefore, the positions of each of the conductive layers 11, 12, and 13 are not limited to the particular embodiment as illustrated in FIG. 1A. In addition, there may be any number of conductive layers and conductive elements in the wearable device 1 based on design requirements.

In some embodiments, the pair of ear hooks may be mounted on and applied to other wearable devices, such as other pairs of glasses, according to the user's needs. For example, the pair of ear hooks (with the conductive layers 11, 12, and 13) can be removed from the wearable device 1. In other words, the pair of ear hooks (with the conductive layers 11, 12, and 13) is detachable. In other words, the pair of ear hooks (with the conductive layers 11, 12, and 13) is replaceable or changeable. Alternatively, the wearable device on which the pair of ear hooks (with the conductive layers 11, 12, and 13) is mounted is replaceable or changeable. This is convenient and cost-effective for the user.

In some embodiments, the conductive layers 11, 12, and 13 may be mounted on the wearable device 1 through other equipment or carriers that can be connected, fixed, or attached to the wearable device 1. In some embodiments, the conductive layers 11, 12, and 13 may be mounted on the wearable device 1 through other equipment that can be detached from the wearable device 1. In some embodiments, the pair of ear hooks may be omitted, and the conductive layers 11, 12, and 13 may be directly integrated in the temples 10 of the wearable device 1. For example, the conductive layers 11, 12, and 13 may be directly formed on the temples 10 of the wearable device 1.

In some embodiments, the conductive layers 11, 12, and 13 may be provided on other portions (such as the nose pads, the bridge, and so on) of the wearable device 1 indirectly (e.g., through the other equipment) or directly (e.g., directly integrated therein) based on the designed requirements of the present disclosure, i.e., electrical performance, cost, or convenience.

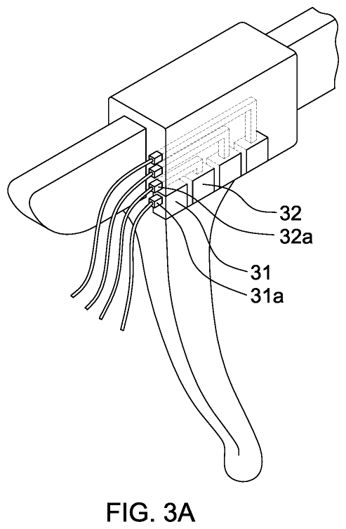

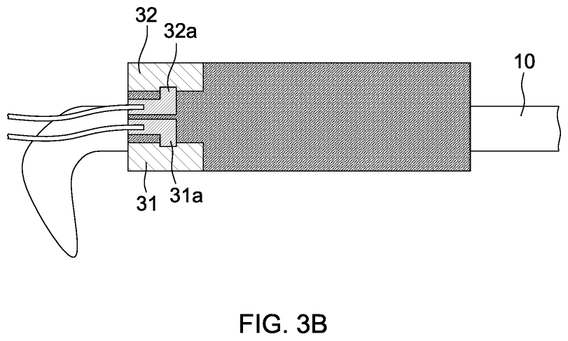

FIG. 3A illustrates a 3D view of an ear hook on a part of a wearable device in accordance with some embodiments of the present disclosure. The ear hook of FIG. 3A is similar to the ear hook of FIG. 1A, and the differences therebetween are described below.

A plurality of conductive layers (including the conductive layers 31 and 32) are provided on the interior surface (i.e., a skin-facing side) of the ear hook. A plurality of conductive elements (including the conductive elements 31a and 32a) is each embedded in one of the conductive layers. The conductive layers may be spaced apart from each other. For example, the conductive layer 31 may be spaced apart from the conductive layer 32 laterally. The conductive layers may be electrically isolated from each other. As such, each of the conductive layers may collect one or more signals (representing bio-signals) of the user without interference from other signals collected via other conductive layers. Different conductive layers may be used to collect different bio-signals associated with the user. In some embodiments, the conductive layers may be used to obtain different signals which represent different bio-signals of the user.

FIG. 3B illustrates a cross-sectional view of a part of a wearable device in accordance with some embodiments of the present disclosure. The part of the wearable device in FIG. 3B is similar to the wearable device in FIG. 3A except for the differences described below.

In some embodiments, as shown in FIG. 3B, the conductive layers 31 may be arranged adjacent to a lower edge of the ear hook and the conductive layers 31 may be arranged adjacent to an upper edge of the ear hook.

In some embodiments, each of the conductive layers and each of the conductive elements may have the similar or the same configurations as shown in FIG. 1A, FIG. 1B, FIG. 1C, FIG. 1D, and FIG. 1E. For example, in some embodiments, the conductive elements 31a may be not embedded in the conductive layer 31, and a surface of the conductive element 31a may be disposed adjacent to a surface of the conductive layer 31.

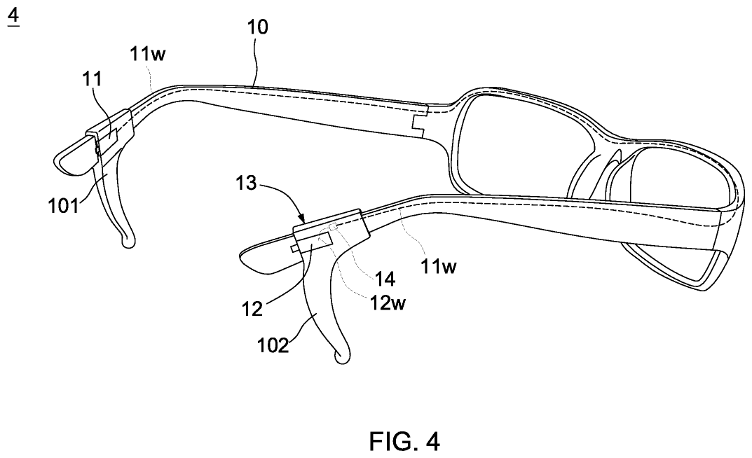

FIG. 4 illustrates a 3D view of a wearable device 4 in accordance with some embodiments of the present disclosure. The wearable device 4 of FIG. 4 is similar to the wearable device 1 of FIG. 1A, and the differences therebetween are described below.

The sensor module 14, and the conductive wires 11w, 12w, and 13w are integrated in the wearable device 4. Therefore, the wearable device 4 is relatively compact and portable. For example, the sensor module 14 may integrated in the temples (or hinges) 10 of the wearable device 4. For example, the sensor module 14 may integrated in the portion 102 (and/or the portion 101) of the wearable device 4. For example, the conductive wires 11w, 12w, and 13w may integrated in the temples (or hinges) 10 of the wearable device 4. For example, the conductive wires 11w, 12w, and 13w may integrated in the portion 102 (and/or the portion 101) of the wearable device 4.

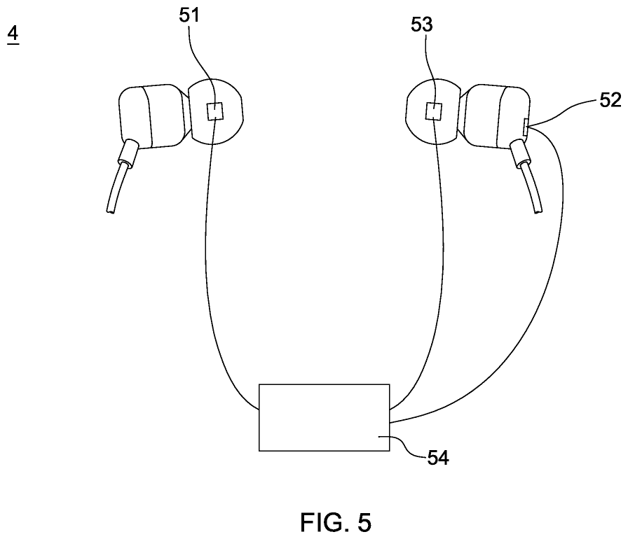

FIG. 5 illustrates a 3D view of a wearable device 5 in accordance with some embodiments of the present disclosure. Examples of the wearable device 5 include a pair of earpieces.

The functions, configurations, and the materials of the wearable device 5 may be similar with the functions, configurations, and the materials as described above with respect to the wearable device 1 and will not be repeated hereafter. For example, the wearable device 5 has conductive layers 51, 52, and 53. The conductive layers 51 and 53 are provided on a support that may be put in (or adjacent to) a user's ear canals. The conductive layer 52 is provided on a housing connecting with the support. When the wearable device 5 is worn by the user, the conductive layers 52 and 53 is on the user's left side and the portion 51 is on the user's right side. The conductive layer 51 may be put in (or adjacent to) a user's right ear canal. The conductive layer 53 may be put in (or adjacent to) a user's left ear canal. The signals from the conductive layers 51, 52, and 53 may be coupled to a sensor module 54 though conductive wires for further processed.

When the user wears the wearable device 5 of FIG. 5, the conductive layer 51 may contact the user's right ear (or a near portion thereof), and the user may contact the conductive layer 52 by the left hand. The conductive layer 51 may receive biological signals (such as ECG signals) from the user's right ear and the conductive layer 52 may receive biological signals (such as ECG signals) from the user's left hand. Thus, the conductive layer 51 and the conductive layer 52 may form a signal loop between the right ear and the left hand. The conductive layer 53 may contact the user to provide a reference for the ECG signals, or may otherwise be used to reject or remove noise from the ECG signals. As another example, the reference conductive layer (such as the conductive layer 53) may be omitted.

In some alternative embodiments, the conductive layer 52 may be integral with the sensor module 54 or a housing containing the sensor module 54. For example, the conductive layer 52 may be disposed on a housing containing the sensor module 54. For example, the conductive layer 52 may be disposed in a housing containing the sensor module 54. For example, the conductive layer 52 may be electrically connected to the sensor module 54. For example, the conductive layer 52 may be received in the housing and can be pulled out of the housing to be brought to be in contact with the user's body. For example, the conductive layer 52 may be received in the housing and can be pulled out of the housing to be close to the user's carotid artery or other portions to detect a biologically-relevant information.

Spatially relative terms, such as “beneath,” “below,” “lower,” “above,” “upper,” “left,” “right” and the like, may be used herein for ease of description to describe one element or feature's relationship to another element(s) or feature(s) as illustrated in the figures. The spatially relative terms are intended to encompass different orientations of the device in use or operation, in addition to the orientation depicted in the figures. The apparatus may be otherwise oriented (rotated 90 degrees or at other orientations) and the spatially relative descriptors used herein may likewise be interpreted accordingly. It should be understood that when an element is referred to as being “connected to” or “coupled to” another element, it may be directly connected to or coupled to the other element, or intervening elements may be present.

As used herein, the terms “approximately”, “substantially”, “substantial” and “about” are used to describe and account for small variations. When used in conduction with an event or circumstance, the terms can refer to instances in which the event or circumstance occurs precisely as well as instances in which the event or circumstance occurs to a close approximation. As used herein with respect to a given value or range, the term “about” generally means within ±10%, ±5%, ±1%, or ±0.5% of the given value or range. Ranges can be expressed herein as from one endpoint to another endpoint or between two endpoints. All ranges disclosed herein are inclusive of the endpoints unless specified otherwise. The term “substantially coplanar” can refer to two surfaces within micrometers (μm) of lying along the same plane, such as within 10 within 5 within 1 or within 0.5 μm of lying along the same plane. When referring to numerical values or characteristics as “substantially” the same, the term can refer to the values lying within ±10%, ±5%, ±1%, or ±0.5% of an average of the values.

The foregoing outlines features of several embodiments and detailed aspects of the present disclosure. The embodiments described in the present disclosure may be readily used as a basis for designing or modifying other processes and structures for carrying out the same or similar purposes and/or achieving the same or similar advantages of the embodiments introduced herein. Such equivalent constructions do not depart from the spirit and scope of the present disclosure, and various changes, substitutions, and alterations may be made without departing from the spirit and scope of the present disclosure.

What is claimed is:

1. A wearable device, comprising:
a first sensing element configured to be disposed adjacent to a right ear of a user while the wearable device is worn by the user;
a second sensing element configured to be disposed adjacent to a left ear of the user and coupled to the first sensing element while the wearable device is worn by the user, wherein the second sensing element and the first sensing element are configured to sense a biological signal from the user; and
a reference electrode configured to reduce an interference to the biological signal.

2. The wearable device of claim 1, wherein the biological signal includes a heart rate variability (HRV).

3. The wearable device of claim 2, wherein the first sensing element and the second sensing element are configured to form a part of an electrocardiogram (ECG) lead.

4. The wearable device of claim 1, wherein the first sensing element is configured to receive a first signal from a first area of a head of the user, the reference electrode is configured to receive a second signal from an second area of the head of the user, and the second sensing element is configured to receive a third signal from a position of a hand of the user.

5. The wearable device of claim 4, further comprising a first support and a second support, wherein the first support is configured to attach the first sensing element to the first area of the head and the second support is configured to attach the reference electrode to the second area of the head, and wherein the reference electrode and the second sensing element are physically disconnected by the second support.

6. The wearable device of claim 5, further comprising a first carrier detachable from the first support and a second carrier detachable from the second support, wherein the first carrier is configured to attach the first sensing element to the first area of the head and the second carrier is configured to attach the reference electrode to the second area of the head.

7. The wearable device of claim 6, wherein the second sensing element and the reference electrode are provided on opposite sides of the second carrier.

8. The wearable device of claim 6, wherein the first carrier is electrically connected with the first sensing element.

9. The wearable device of claim 8, wherein the second carrier is electrically connected with the reference electrode.

10. The wearable device of claim 5, further comprising a sensor module connected to the first support or the second support, wherein the sensor module is coupled to the first sensing element, the second sensing element, and the reference electrode, and the sensor module is configured to determine the biological signal from the first signal, the second signal, and the third signal.

11. The wearable device of claim 10, wherein the wearable device comprises a pair of glasses, the sensor module is integrated with the pair of glasses, the first support and the second support comprises hinges of the pair of glasses, wherein the pair of glasses includes an embedded conductive wire extending between the first support and the second support, wherein the embedded conductive wire is connected with the sensor module, the first sensing element, the second sensing element, and the reference electrode.

12. The wearable device of claim 5, wherein the wearable device comprises a first earphone and a second earphone, the first earphone comprises a first housing connecting the first support configured to match a left ear canal, and the second earphone comprises a second housing connecting the second support configured to match a right canal, and the second sensing element is disposed on the second housing.

13. The wearable device of claim 12, further comprising a sensor module integrated with the first housing or the second housing and coupled to the first sensing element, the second sensing element, and the reference electrode, and wherein the sensor module is configured to determine the biological signal from the first signal, the second signal and the third signal.

14. The wearable device of claim 6, wherein the first sensing element is partially embedded in the first carrier, and the reference electrode and the second sensing element are partially embedded in the second carrier.

15. The wearable device of claim 12, wherein the first sensing element is partially embedded in the first support, and the reference electrode is partially embedded in the first support.

16. The wearable device of claim 10, the sensor module comprises a biological signal amplifier electrically connected with the referenced electrode to reduce a common-mode interference.

17. A headset device, comprising:
a first sensing element configured to contact a first position of a user's head and receive a first electrical potential from the first position when the headset device is worn by the user; and
a second sensing element configured to receive a second electrical potential from a second position of the user's body, wherein the second position is different from the first position.

18. The wearable device of claim 17, further comprising a processor configured to determine a biological signal from the first electrical potential and the second electrical potential.

19. The wearable device of claim 17, wherein the first position is on the right of the user's head, the second position is on the left of the user's body.

20. A method for operating a wearable device, comprising:
contacting a first conductive layer by a first position of a user's head to receive a first signal from the first position; and
contacting a second conductive layer by a second position of the user's body to receive a second signal from the second position.
